(19) United States
(12) Patent Application Publication
(10) Pub. No.: US 2012/0123397 A1
(43) Pub. Date: May 17, 2012

(54) METHOD AND APPARATUS FOR LIPOSUCTION

(76) Inventors: Haim Epshtein, Benyamina (IL); Boris Vaynberg, Zikron Yaakov (IL); Ganady Nahshon, Binyamina (IL)

(21) Appl. No.: 13/262,368

(22) PCT Filed: Mar. 18, 2010
(86) PCT No.: PCT/IL10/00222

§ 371 Date: Nov. 1, 2011

Related U.S. Application Data
(60) Provisional application No. 61/165,529, filed on Apr. 1, 2009.

(51) Int. Cl. A61B 18/20; A61B 18/14

(52) U.S. Cl. 606/10

(57) ABSTRACT

A controller, an external electrode and a probe to be inserted into a tissue volume. One end of the probe is terminated by one or more electrodes and the other end is connected to a load cell. The electrode end of the probe is inserted into a volume of adipose tissue to be treated. Intermittently different and low power RF energy is applied between the external electrode and the electrodes of the probe. Monitoring of the impedance between the external electrode and the probe electrodes enables probe guidance. Monitoring of the impedance between the probe electrodes helps characterize the aggregate state of the treated tissue volume and treatment end-point.

### BACKGROUND

The present device, apparatus, and method relate to the field of adipose tissue treatment and aesthetic body sculpturing.

Liposuction is a technique for removal of fat from different sites of the subject and, in particular, the subject body. The process changes the external contours of the body and is sometimes described as body sculpturing. The fat or adipose tissue is removed by a suction device via a cannula inserted into the appropriate site of the body. The process is painful and sometimes causes excessive bleeding.

Recently, liposuction procedures have been improved by the use of electro-magnetic radiation such as an infrared laser radiation delivered through a fiber inserted into a cannula introduced into the treatment site. Laser radiation liquefies the adipose tissue. The liquefied tissue is either removed by suction or left in the subject body, where it gradually dissipates in a uniform way. Laser assisted liposuction is considered to be a more advanced and less invasive procedure when compared to traditional liposuction techniques.

For proper treatment, laser assisted liposuction requires application of high power, ten to fifty watt, laser radiation. The radiation is applied in continuous or pulse mode for relatively long periods with the help of a fiber the tip of which is introduced into the adipose tissue. Frequently, more than one laser is used on the same treated tissue volume to speed up the treatment. Each of the lasers may operate in a different mode. For example, one of the lasers preheats the target tissue volume, and the other one introduces laser power sufficient to destroy the adipose tissue in the same volume. Laser assisted liposuction requires frequent cleaning and maintenance of the fiber tip from process debris. All of the above slows down the treatment process, affects comfort and cost of the procedure to the treated subject.

Sometimes liposuction is conducted by application of radio frequency (RF) energy to the adipose tissue. A probe with electrodes is introduced subcutaneously, moved back and forth, and depending on the level of RF energy, it affects a volume of adipose tissue or fat larger than the one affected by the laser radiation. This accelerates the liposuction process and shortens the treatment period

In both laser and RF assisted liposuction treatments, location of the subcutaneously introduced probe is not known. The treatment provider may occasionally push the probe into a muscle or skin overlaying the treated tissue volume and damage them.

The present method provides a better than currently available solution to these and other existing liposuction problems.

### GLOSSARY

The term “monopolar configuration” as used in the present disclosure means a configuration consisting of an active treatment electrode and a passive one, which acts as the grounding electrode. Typically the electrodes are different in size and located at a distance between them. RF induced current affects the tissue area/volume that is proximate to the active electrode.

The term “bipolar configuration” as used in the present disclosure means that the current passes between two identical electrodes located a short distance apart from each other. They are applied to the area/volume of tissue to be treated and the propagation of the current is limited to the area between the electrodes themselves.

The terms “skin” as used in the present disclosure means the outer skin layers such as stratum corneum, dermis, epidermis, etc.

The terms “tissue”, “fat” or “adipose tissue” as used in the present disclosure have the same meaning and are used interchangeable through the text of the disclosure.

The term “tissue affecting energy” as used in the present disclosure means energy capable of causing a change in the tissue and/or skin or enabling such change. Such energy for example, may be RF energy, optical radiation in visible or invisible part of electromagnetic spectrum, ultrasound waves energy, and kinetic energy provided by a massaging device.

The term “probe” as used in the present disclosure means any device operative to couple to the tissue or skin energy affecting the tissue/skin. Such device for example, may apply to the tissue RF energy, optical radiation existing in the visible or the invisible part of spectrum, ultrasound waves energy, kinetic energy provided by a massaging device or some other source of energy.

As used herein, the terms “person” and “subject” have the same meaning and refer to any human or animal subject, as well as synthetic objects.

As used herein, the terms “optical radiation sources” and “optical radiation emitters” have the same meaning and refer to any source or emitter of visible or non-visible optical radiation.

As used herein, the term “tumescent solution or fluid” means a solution of sterile dilute salt water, adrenaline, lidocaine, anesthetic material, and some other ingredients injected into the adipose tissue and ballooning the target volume of adipose tissue, allowing easier penetration of the probe and adipose tissue treatment products removal.

As used herein, the term “treatment” means a process of coupling to the tissue or skin energy affecting the tissue/skin.

### BRIEF SUMMARY

A method and apparatus for guiding a probe delivering tissue treatment energy in laser and RF energy assisted liposuction treatment. The apparatus includes a controller; an external electrode and a probe operative to be inserted into a tissue volume. The probe is a light guide terminated on one of it ends by one or more electrodes. The opposite end of the probe is connected to a load cell. A treatment provider penetrates the terminated by electrodes end of the probe into a volume of adipose tissue to be treated and applies to it laser radiation and/or treatment RF energy. Intermittently different and low power RF energy is applied between the external electrode and the electrodes terminating the probe. Monitoring of the impedance between the external electrode and the electrodes terminating the probe enables probe guidance. Monitoring of the impedance between the electrodes terminating the probe enables determination of the aggregate state of the treated tissue volume and treatment end-point. Load cell signal provides additional information on the type of tissue treated and facilitates advance of the probe.

### BRIEF LIST OF DRAWINGS

The disclosure is provided by the way of non-limiting examples only, with reference to the accompanying drawings, wherein:

FIG. 1A is a schematic illustration of an exemplary embodiment of the present apparatus for liposuction.

FIG. 1B is a schematic illustration of an exemplary embodiment of the probe of the present apparatus for liposuction.

FIGS. 2A through 2H are schematic illustrations of some exemplary embodiments of the elongated light conducting body of the probe and associated with it electrodes.

FIGS. 3A and 3B are schematic illustrations of exemplary embodiments of the external electrode.

FIG. 4 is a graph illustrating the impedance of the different types of tissue.

FIG. 5 is a schematic illustration of tissue treatment employing the present apparatus.

FIG. 6 are schematic illustration of the method of diagnosing the type of the treated tissue.

FIG. 7 is a schematic illustration of an exemplary embodiment of treatment and diagnostics signals sequence of the present method for RF and laser assisted liposuction.

FIG. 8 is a schematic illustration of an exemplary embodiment of the method of advancing the treatment probe in the treated tissue.

FIG. 9 is a schematic illustration of the load cell readings when the probe is located in different types of tissue.

FIG. 10 is a schematic illustration of the adipose tissue treatment in presence of tumescent liquid.

FIG. 11 is a schematic illustration of the treatment end-point determination according to one exemplary embodiment of the present method for RF and laser assisted liposuction.

### DETAILED DESCRIPTION

In the following detailed description, reference is made to the accompanying drawings that form a part hereof and wherein like reference numerals denote like elements through the several views.

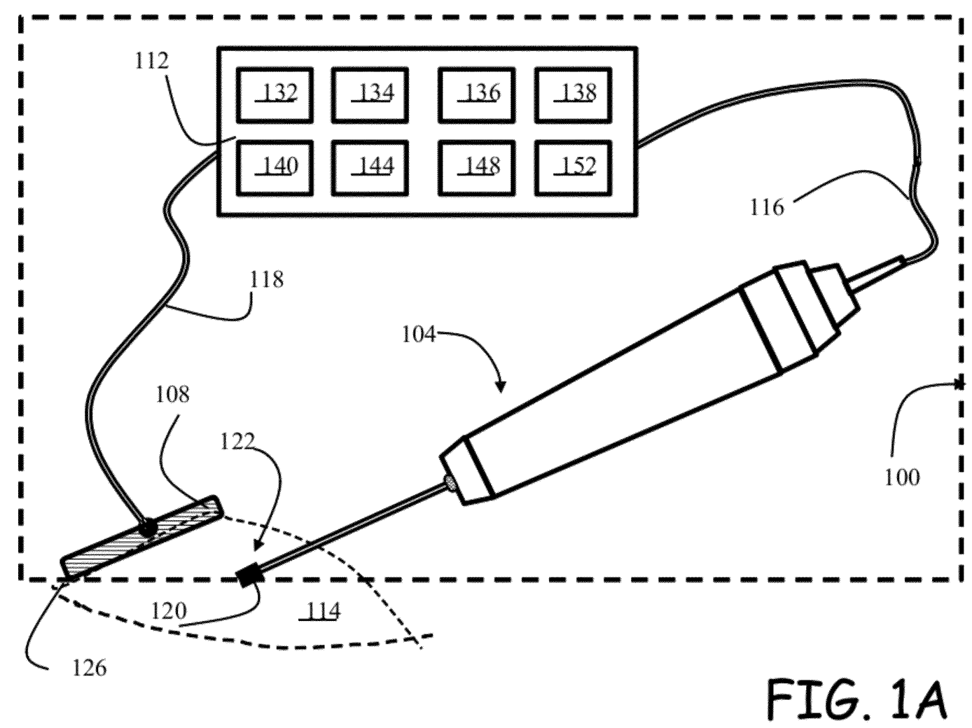

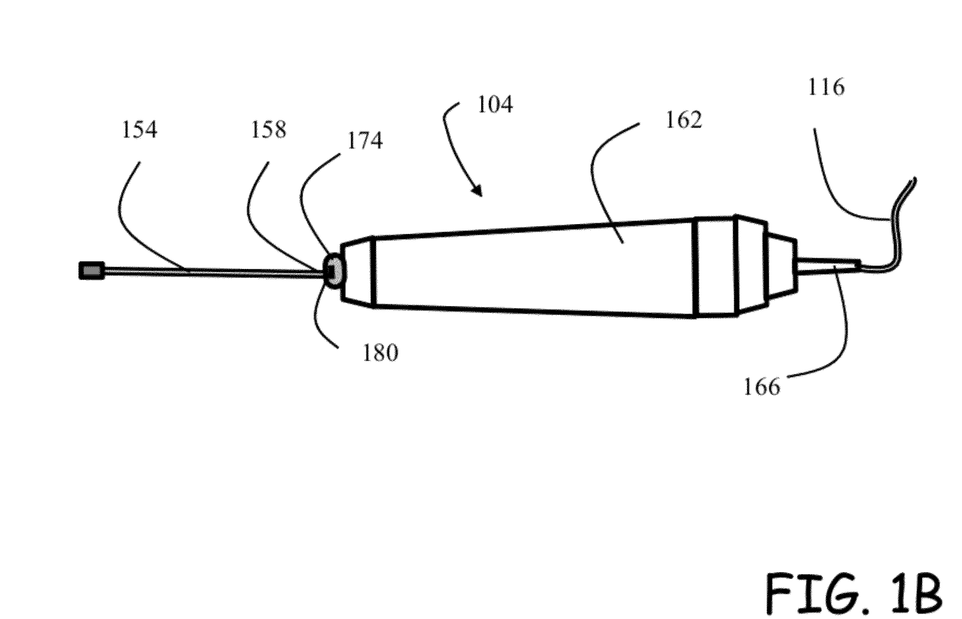

Reference is made to FIG. 1A, which is a schematic illustration of an exemplary embodiment of an apparatus for cosmetic and body shaping procedures and in particular for RF and laser assisted liposuction. Apparatus 100 includes a probe 104, an external electrode 108, a controller 112, and cables 116 and 118 connecting probe 104 and electrode 108 to controller 112. In operation, cable 116 enables electric, fluid, and optical communication between controller 112 and probe 104. Cable 118 enables electric communication between controller 112 and external electrode 108. One or more electrodes 120 terminate the distal end 122 of probe 104, which in operation is inserted into a volume of tissue 114 schematically shown by phantom lines. The external electrode 108 is configured in course of operation to be in permanent contact with a skin surface 126 and may be applied to any segment of the skin surface of a treated subject. Electrode 108 typically would have an area substantially larger than the one or more electrodes 120 terminating probe 104.

Controller 112 includes a source of low RF voltage or power 132, a source of high RF voltage or power 134, a source of laser radiation 136, and a mechanism 138 for monitoring impedance between the external electrode 108 and the electrode 120 terminating the distal end 122 of probe 104. The source of low RF power or voltage 132 provides a low RF power voltage, which is used primarily for diagnostic purposes. The diagnostic RF or low power voltage may be supplied in a mono-polar mode between the external electrode 108 and one of the electrodes 120 located on the distal end 122 of the inserted in the tissue probe 104. The source of high RF power 134 provides a RF power level sufficient to cause a desired tissue treatment effect. The high power RF voltage is typically supplied in a bi-polar mode between electrodes 120. In order to reliably distinguish between the low RF voltage and the high power RF voltage one of the voltages may have at least one different parameter. The parameter may be the RF voltage amplitude, RF voltage frequency, operation time etc. Both of the RF voltage sources 132 and 134 may supply the RF voltage or energy in a pulse mode or in a continuous mode.

Controller 112 may include one or more sources 136 of laser radiation. Alternatively, the laser radiation sources may be stand-alone units. For example, a high power NeYAG laser may be used to cause the desired tissue treatment effect. Additional laser sources emitting radiation with wavelength favorably affecting or causing blood vessels coagulation and/or charring may be conducted through the same probe 104. The source of laser radiation may supply the laser radiation in a pulse mode or in a continuous mode.

Controller 112 may include a processor 140 synchronizing and controlling operation of the apparatus 112 and a cooling fluid supply pump 144 or other source of cooling fluid. Controller 112 may optionally include a mechanism for impedance monitoring between electrode 108 and at least one of the electrodes 120 located on the distal end 122 of probe 104, and in cases where electrode 120 is more than one electrode between the electrodes forming the electrode 120. In some embodiments the impedance monitoring mechanism between electrodes 120 located on the distal end 122 of probe 104 may be different of separate mechanisms. The treatment parameters and the treatment progress may be displayed on a display 148. A keypad 152 or touch type display may be used for setting of the treatment parameters.

FIG. 1B is a schematic illustration of an exemplary embodiment of the probe 104. Probe 104 typically includes an elongated light conducting body 154 and a handle 162. In one embodiment, the proximal end 158 of the elongated light conducting body 154 is permanently connected to handle 162 facilitating probe handling. A cable connector 166 coupling with cable 116 may terminate the other end of handle 162. Cable connector 166 is adapted to receive in operation one of more types of RF voltages generated by RF voltage sources 132 and 134 (See FIG. 1A) and laser radiation proved by the source of laser radiation 136.

In another exemplary embodiment, the proximal end of the elongated light conducting body 154 is releasable connected to handle 162 for example, with the help of connector 174 such that when necessary it may be fast and easy replaced by another elongated light conducting body 154. In use the connector 174 receives one or more types of RF voltages generated by the RF voltage sources 132 and 134 (FIG. 1A) and laser radiation provided by the source of laser radiation 136. A load cell 180 may be incorporated in the connector or configured to connect the elongated light conducting body 154 to handle 162.

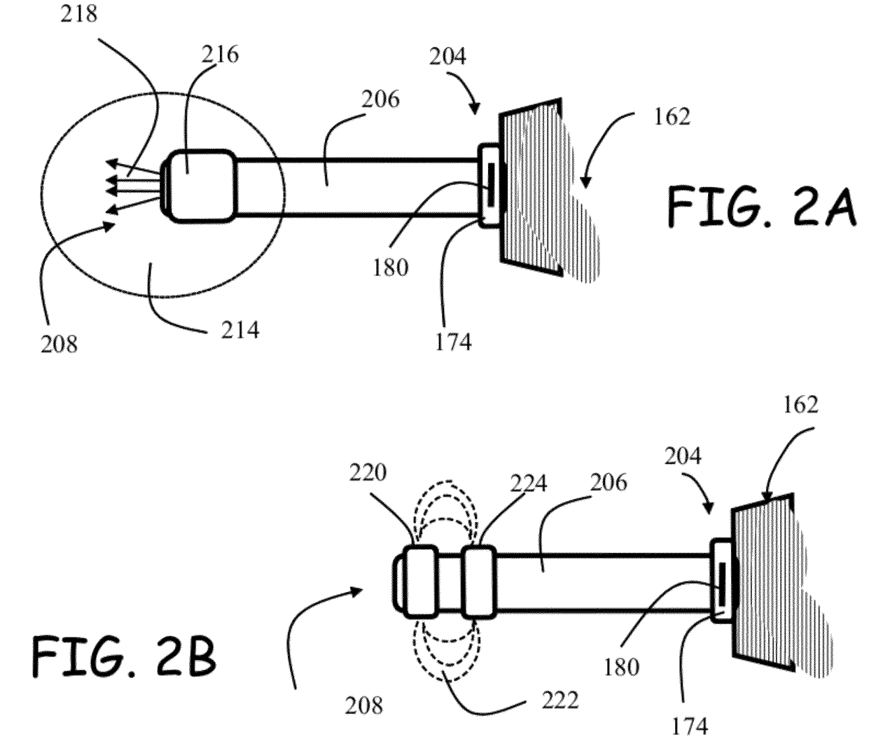

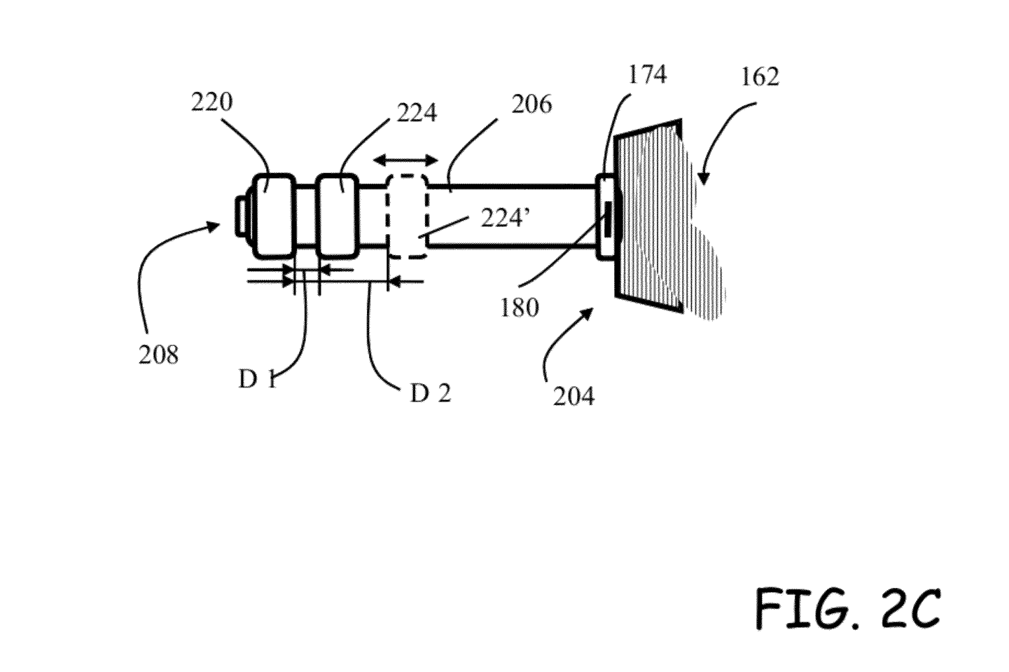

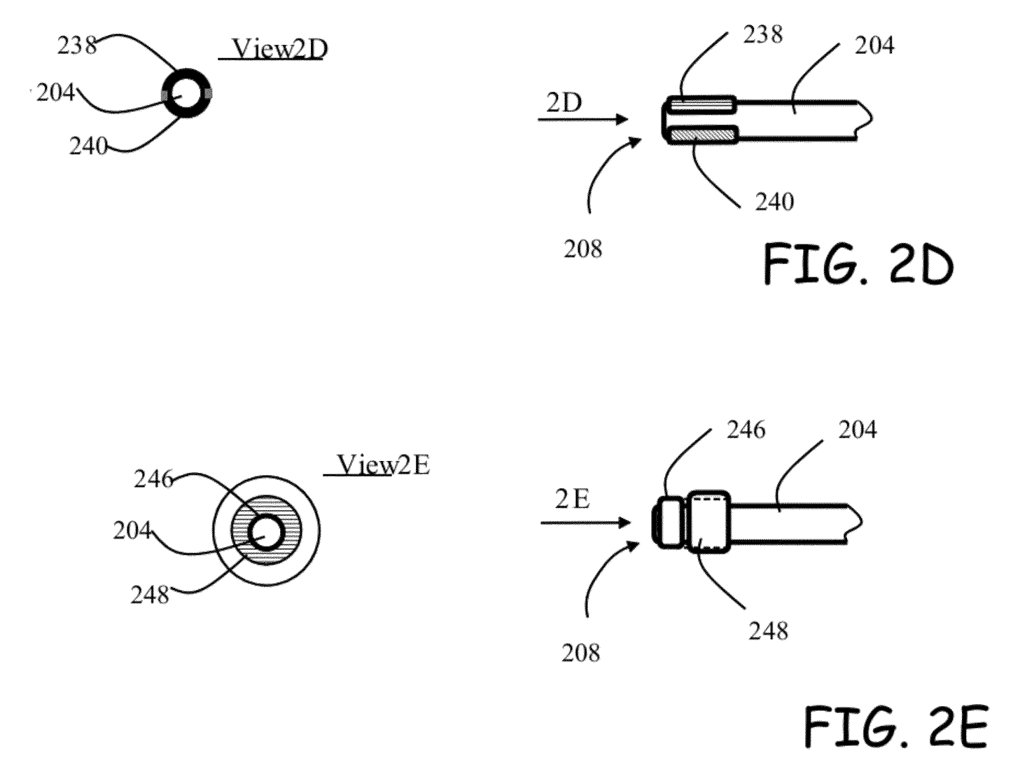

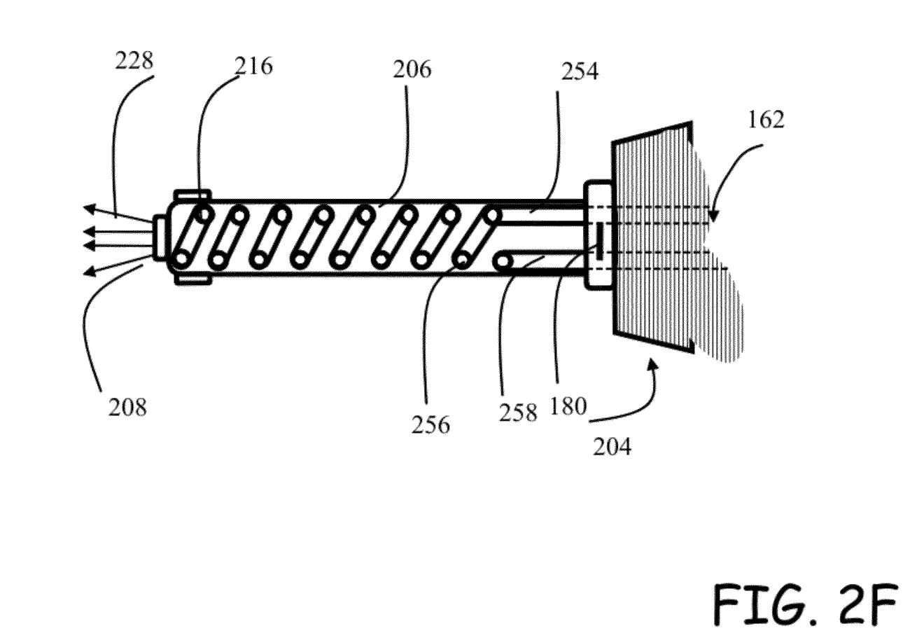

FIGS. 2A through 2F provide addition details of some exemplary embodiments of the probe. Probe 204 includes an elongated laser radiation conducting body 206. In operation the distal end 208 of the body 206 is inserted into a volume of tissue 214 shown by phantom lines. Laser radiation emitted through the end of the elongated body 206 is illustrated by laser radiation marks 218. The radiation is emitted into the treated volume of tissue 214. In one exemplary embodiment, the distal end 208 of the elongated light conducting body 206 is terminated by one electrode 216 (FIG. 2A). In another embodiment, illustrated in FIG. 2B the distal end 208 of the elongated light conducting body 206 or probe 204 is terminated by a pair of electrodes 220 and 224. The pair of electrodes 220 and 224 may be arranged to change the distance between them as shown by D1 and D2 (FIG. 2C) and displaced electrode 224′. The proximal end of light conducting body 206 may be connected to handle 162 with the help of connector 174 that may include load cell 180 (FIG. 1). Alternatively, load cell 180 may be independent from connector 174. The pair of electrodes terminating the probe may be cylindrical electrodes 220 and 224 (FIG. 2B), segments of a cylindrical electrodes 238 and 240 (FIG. 2D), and concentric or coaxial electrodes 246 and 248 (FIG. 2F).

In use the distal end 208 of probe 204 or of the elongated body 206 (FIG.2) applies RF voltage and other tissue treatment energies to the tissue. The heat developed in course of treatment may heat the distal end of the probe, cause uneasiness to the treated subject, cause probe end carbonization, and affect negatively the treatment process. In an additional embodiment (FIG. 2F), probe 204 may be configured to include cooling fluid or gas conducting channels 256 enabling effective cooling fluid delivery and cooling of the distal end 208 of the probe 204. The fluid delivery channel 254 and the fluid return channel 258 may be embedded in the connector 174 and in the probe handle 162. Cable connector 166 (FIG. 1B) and cable 116 are all designed to enable cooling fluid or gas supplied by pump 144 to the distal end 208 of probe 204.

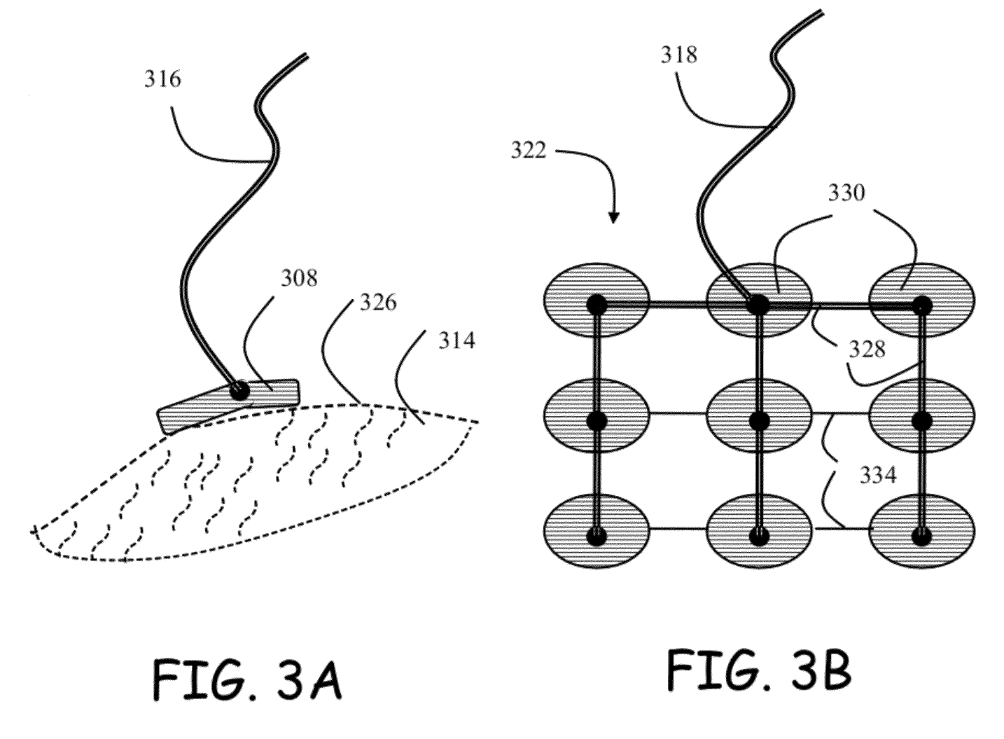

FIGS. 3A and 3B are schematic illustrations of exemplary embodiments of the external electrode. Electrode 308 is sometimes termed a return electrode; it is connected to controller 112 by external electrode cable 316 and is configured in use to be in permanent contact with skin surface 326. The external electrode 308 typically has an area substantially larger than one or both electrodes 220 and 224 (FIG. 2B). The large contact area of the external electrode provides substantial contact with the skin surface 326. The large contact area enables continuous and relatively uniform electric conductivity between the external electrode 308 and the one or both of the electrodes 220 and 224 located at the distal end of the probe 204 (FIG. 2), during treatment of the volume of tissue 314. The external electrode 308 may be implemented as a rigid electrode 108 (FIG. 1A) or flexible electrode 308, or as a grid 322 of electrically interconnected by conductors 328 electrodes 330. Additional electric or non-electric connecting links 334, made from allowed to use for cosmetic skin treatment purposes material, may be provided between electrodes 330 simplifying electrode 322 handling.

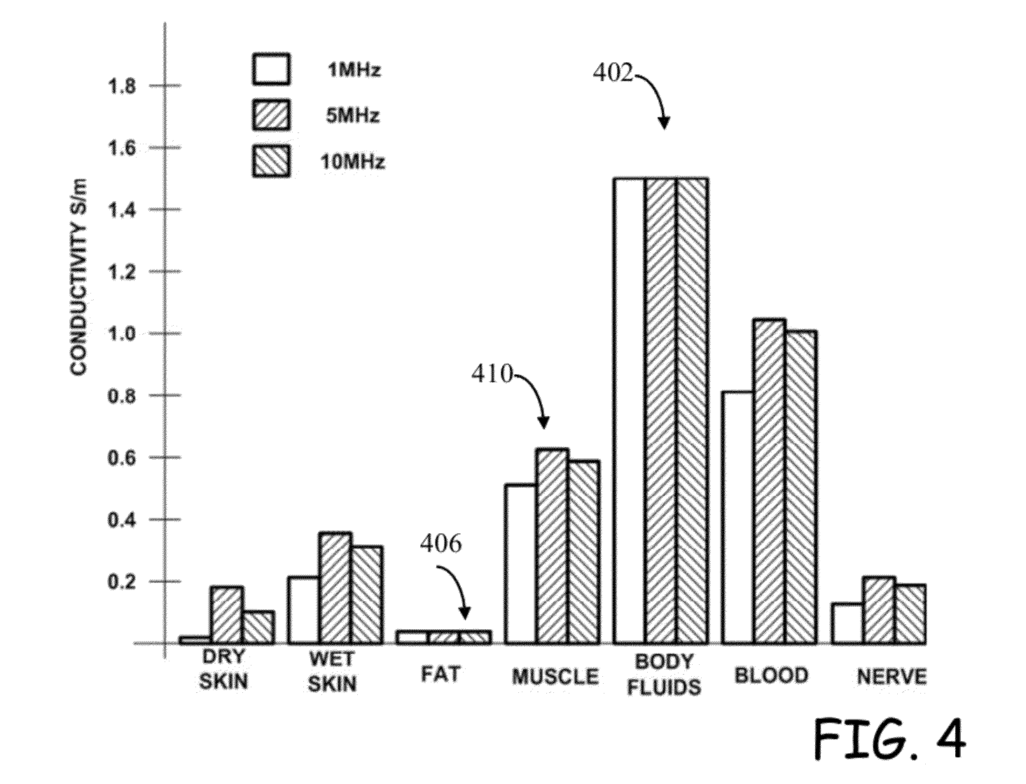

The authors of the present method have measured impedance of different types of tissue and have experimentally found that each of the tissue types has different and sometimes significantly different from the other type of tissue impedance values. FIG. 4 is a schematic illustration of different types of tissue impedance value represented by tissue conductivity measurement results. The graphs indicate that body fluid conductivity 402 is about 50 higher than the conductivity of the adipose tissue or fat 406 and the conductivity of muscle tissue 410 is about 20 times higher than the conductivity of the adipose tissue 406.

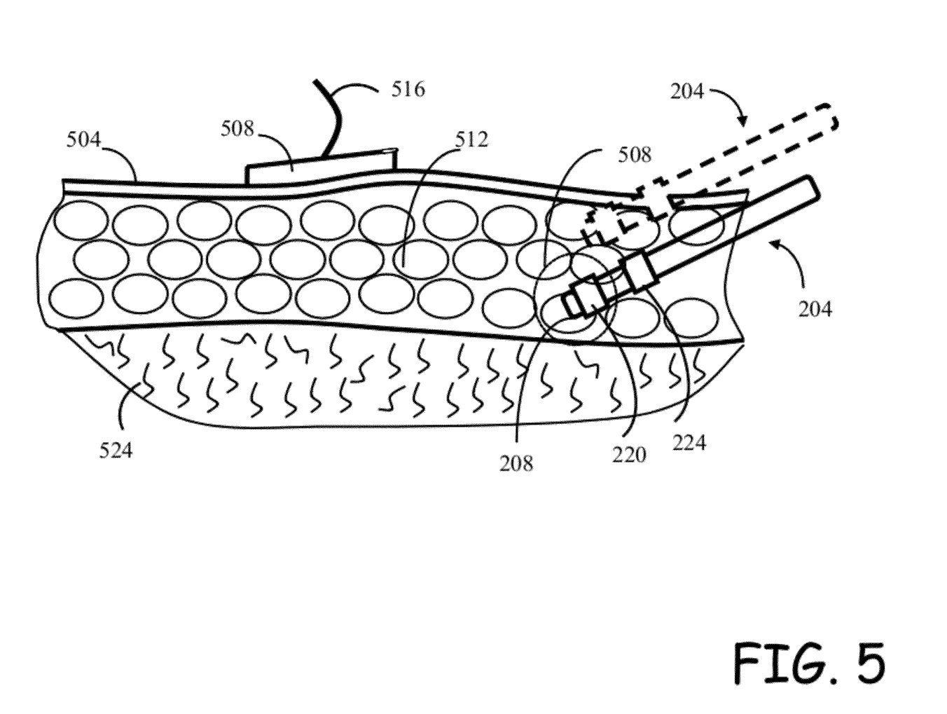

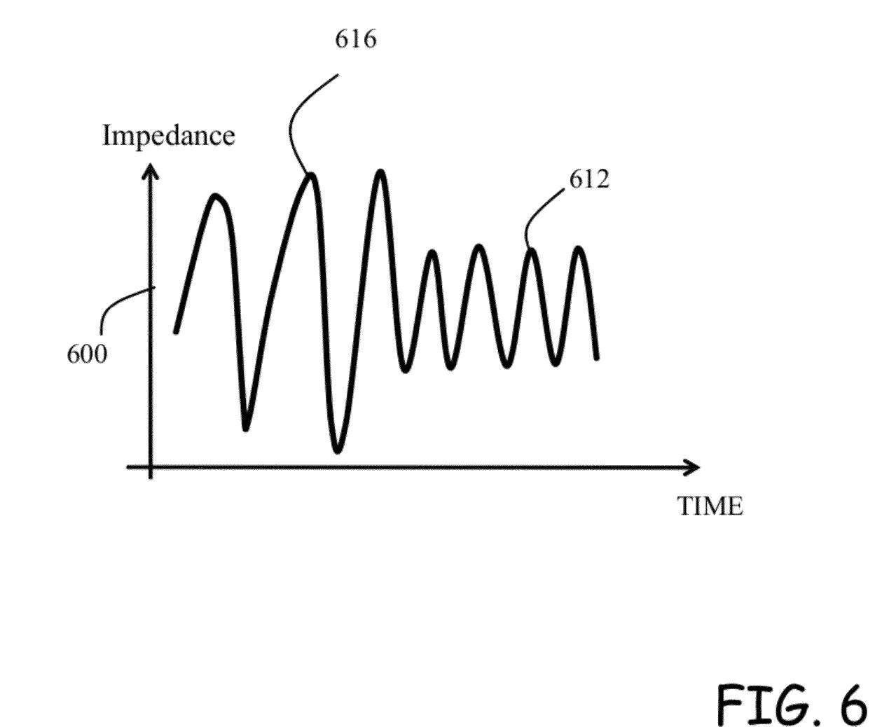

FIG. 5 is a schematic illustration of a tissue treatment process employing the present apparatus. According to the present method of treatment the caregiver applies probe 204 to skin surface 504, pierces it, and penetrates the distal end 208 of the probe 204 into a volume of adipose tissue to be treated 508. The probe penetration into the adipose tissue may be through an incision made in the skin surface 504 by conventional means such as a surgical scalpel or by operating a laser energy source 136, or RF energy source 134, or both of them (FIG. 1) at power level sufficient to make an incision in the skin surface 504 and penetrate into adipose tissue 512. The depth of the probe 204 introduction and magnitude of the tissue affecting energy determine the tissue volume affected by the tissue affecting radiation supplied by sources 134 and 136 (FIG. 1). Controller 112 initiates supply of tissue affecting energy, which may be laser radiation, RF energy, or both of them and the treatment provider or caregiver moves the probe 204 back and forth liquefying the adjacent to the distal end 208 of the probe 204 adipose tissue.

Concurrently with the beginning of the treatment or before the beginning of the treatment an external electrode 508 is applied to a segment of skin surface such as to maintain a permanent contact between the external electrode 508 and the skin surface 504. Low power RF voltage source 132 is activated and applies a low power RF signal between one or more electrodes 220 and 224 located on the distal end 208 of the probe 204 and the external electrode 508 connected with the help of cable 516 to the controller 112. The impedance monitoring mechanism 138 monitors the impedance between the external electrode 516 and one or both electrodes 220 and 224 that for measurement purposes may be temporarily short circuit and maintained under an equal potential, located on the distal end 208 of the probe 204.

According to the present method by monitoring the impedance, between the external electrode 508 and at least one of the treatment electrodes 220 or 224 located on the distal end 208 of the probe 204, it is possible to establish the type of the treated tissue and use this information to guide the probe 204 in the tissue volume. For example, a high impedance value indicates on probe location proximate to adipose tissue or fat 512 and a low impedance value indicates on probe location proximate to the muscular tissue 524. If desired different types of the tissue and probe location may be schematically displayed on display 148 (FIG. 1). As the probe 204 is advanced in the treated tissue 512 the variation from a high impedance value to a low impedance value indicates shift from the adipose tissue 512 to the muscular tissue 524 and the shift from low impedance value to high impedance value indicates shift from muscular tissue 524 to the adipose tissue 512. Thus the method disclosed may be used for guiding the probe delivering tissue treatment energy in cosmetic and body shaping procedures.

The tissue is not a homogenous medium and as the probe 204 advances even in the same type of tissue the measured impedance varies. FIG. 6 shows a graph of the variations of the impedance 600 measured between at least one electrode 220 or 224 located on the distal end of the probe 204 and the external electrode 516. The impedance variations are substantially lower for the muscular tissue 524 (Section 612 of the graph.) than for adipose tissue 512 (Section 616 of the graph.). Application of bi-polar RF voltage requires presence of two relatively closely spaced electrodes 220 and 224. In order to avoid erroneous impedance measurement between the external electrode 316 and the pair of electrodes 220 and 224 they may be maintained under equal RF potential or are short circuit at the time of measurement.

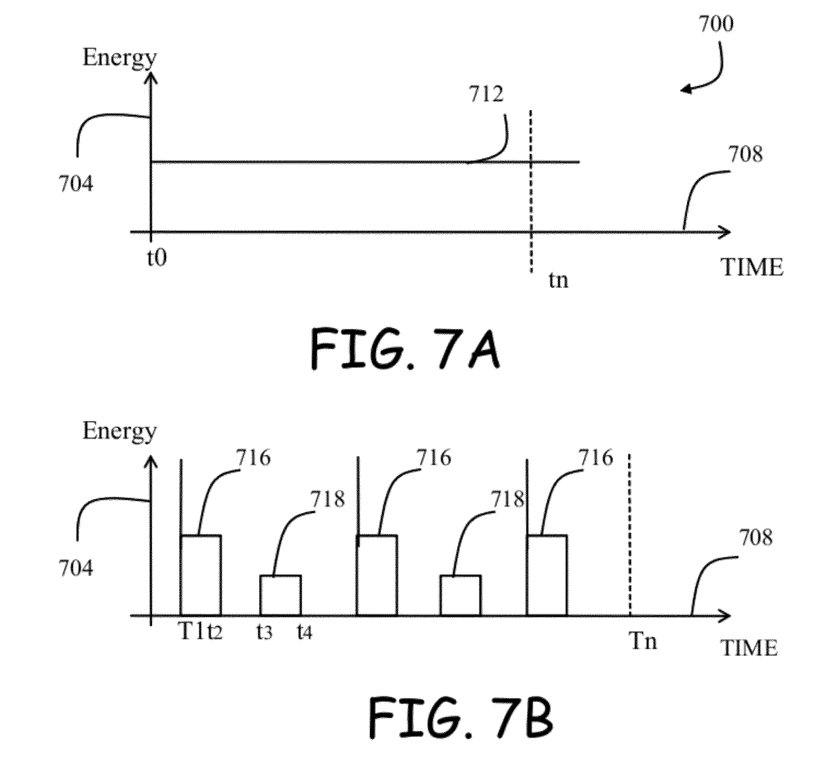

Both the high power RF voltage and the laser radiation affecting the tissue 528 (FIG. 5) according to the method may be supplied in a pulse mode or in a continuous mode. FIGS. 7A and 7B are graphs illustrating the continuous and pulse operation modes. The energy magnitude 704 is plotted over a time scale 708. In the current disclosure continuous 712 means application periods substantially longer than the duration of the treatment pulses 716. The low power RF voltage 718 may be applied in the intervals between the high power RF voltage pulses avoiding any interference between the two RF voltages.

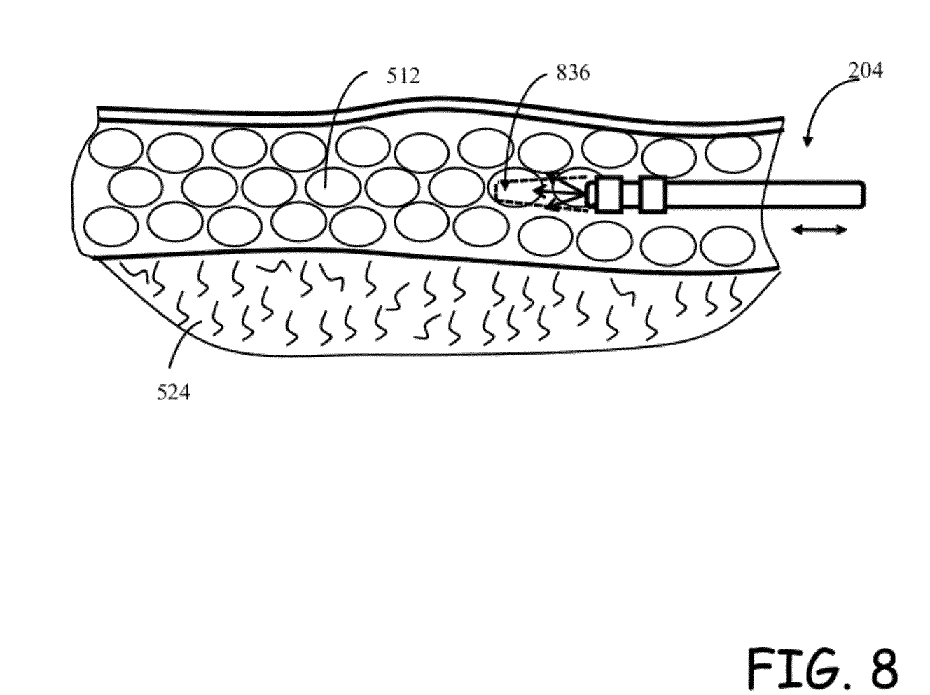

The liposuction treatment (FIG. 8) is performed by moving back and forth inserted into the tissue probe 204. Periodically, the angle of movement is changed to affect additional tissue volumes and also certain anesthetic means are introduced into the tissue, the probe 204 movement causes certain discomfort and pain to the treated subject. According to the present method following the penetration of the probe into the tissue in its movement inside the tissue is facilitated by application to the tissue through the probe 204 (FIG. 2) of laser radiation with a power sufficient to liquefy the adipose tissue. Force resisting mechanical probe movement in the liquefied adipose tissue is substantially lower than of non-liquefied adipose tissue. The liquefied by the laser radiation adipose tissue, is forming a pass 836 enabling easy probe into the tissue penetration. As the probe advances a high power RF energy or voltage may be concurrently applied to the same or adjacent volume of the adipose tissue liquefying a larger volume of the adipose tissue. The laser radiation wavelength may be selected such that in addition to liquefying the adipose tissue it will by coagulating blood vessels damaged in the course of treatment. Alternatively, two laser wavelength of different power may be conducted through the same light guide. One laser wavelength would be selected to be optimal for liquefying the adipose tissue and the other one to coagulate blood vessels.

As disclosed above the apparatus includes a mechanism 138 (FIG. 1) operative to monitor the impedance between the pair of electrodes 220 and 224 (FIG. 2). The impedance between the pair of electrodes 220 and 224 is dependent on the temperature of the treated volume. Higher temperature for example 40 to 60 degrees Celsius indicates presence of liquefied adipose tissue in the treated volume and accordingly on the aggregate state of the treated tissue.

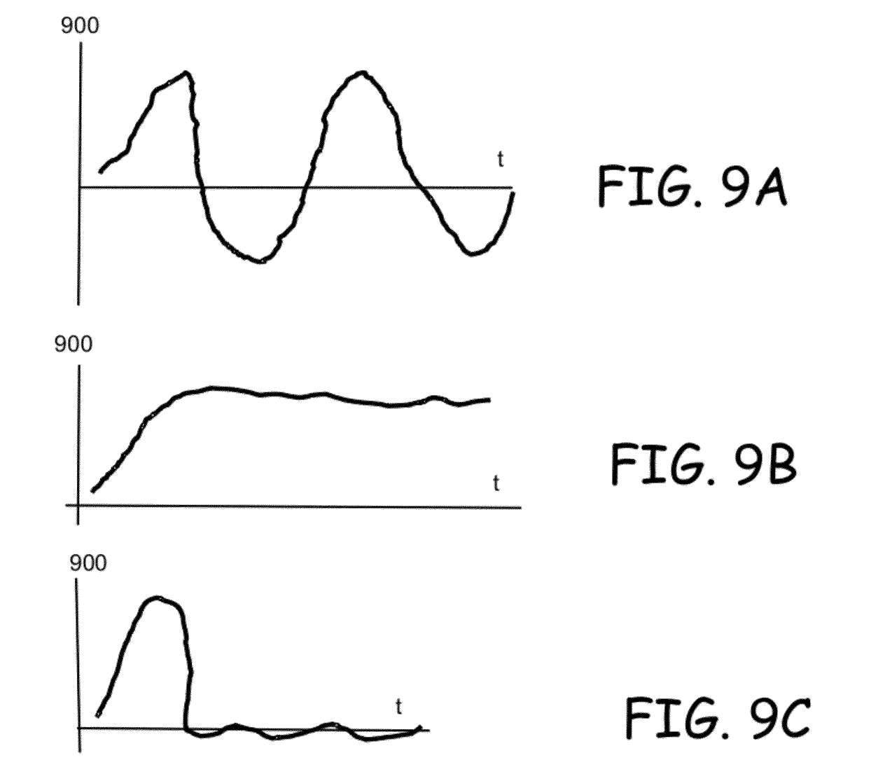

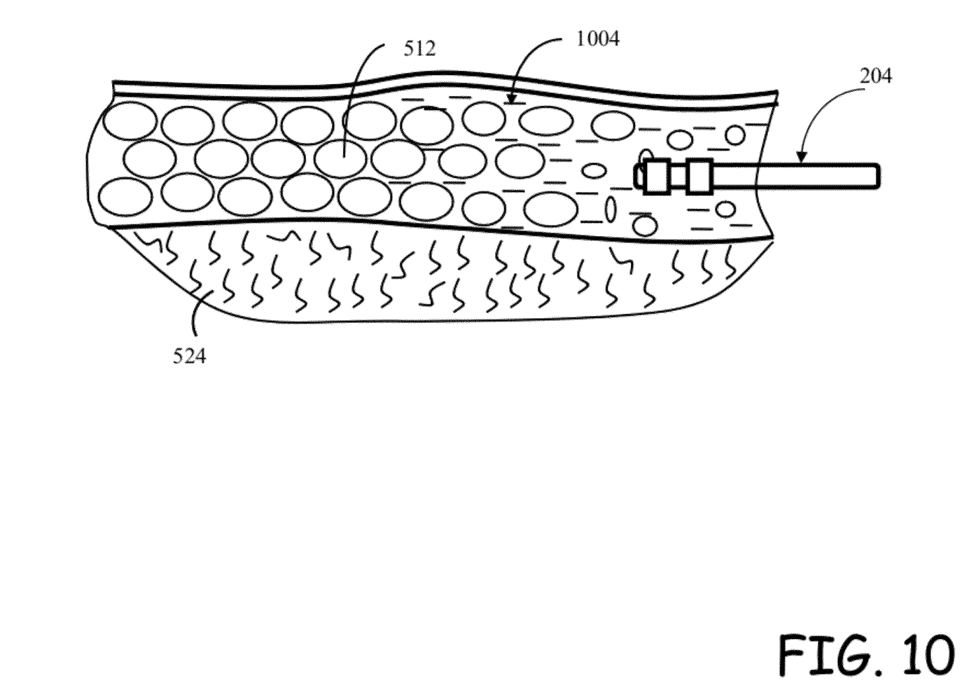

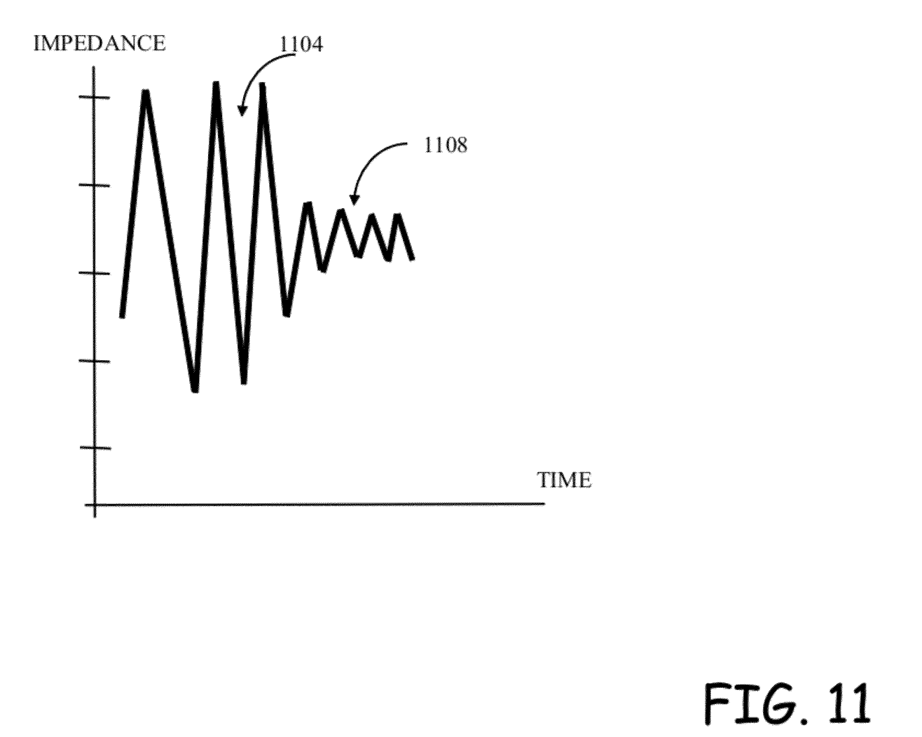

As noted above the liposuction treatment is performed by moving back and forth inserted into the tissue probe 204 and applying to it proper energy. The adipose tissue has a different from the muscle resistance to the mechanical movement of the probe 204. Load cell 180 connected mechanically to probe 204 allows tracking of the adipose tissue and muscle resistance and judgment of the type of tissue treated. FIG. 9 is a schematic illustration of the load cell readings when the probe is located in different types of tissue. Vertical axis 900 indicates load cell reading, where the horizontal axis is the time. FIG. 9A illustrates readings of the load cell 180 when probe 204 moves forward (for example, graph section above the time axis) and backward (for example, graph section below the time axis). Mechanical resistance to probe advance of adipose tissue is different from the resistance of muscle and when the probe approaches muscle the resistance grows (FIG. 9B) and remains almost constant as long as the probe is pushed against the muscle. When probe 204 is pushed against muscle the tissue treatment energy may damage the muscle. Based on probe readings it is possible to switch off tissue treatment energy delivery to the treated tissue volume. When the probe movement is discontinued, there are no forces affecting the probe and the load cell readings become close to zero (FIG. 9C).

It is established to conduct RF based liposuction in presence of tumescent solution or fluid 1004, which is introduced into the tissue prior to the treatment. Tumescent solution has conductivity substantially higher than the adipose tissue, allows faster temperature rise and facilitates the liquefying of the adipose tissue. As the adipose tissue is liquefied or almost liquefied by the applied treatment radiation, pockets of adipose tissue mixed with the tumescent fluid are formed in the treated volume. The difference between the impedance of the tumescent solution and adipose tissue is in order of a number of magnitudes. As the probe advances or the concentration of the treated volume changes the mechanism 138 operates to monitor the impedance between the pair of electrodes 220 and 224 may sense sudden high magnitude impedance variations caused by entry of the pockets of adipose into the treated volume filled by the tumescent fluid.

FIG. 11 is a schematic illustration of the treatment end-point determination according to one exemplary embodiment of the present method for RF and laser assisted liposuction. The treated volume impedance, and in particular the impedance variations in segment 1104, can be used as the liposuction progress indicator. For example, when the magnitude of impedance variations between the pair of electrodes located in the tumescent environment of the treated tissue becomes low as shown in segment 1108 it indicates that almost all of the adipose tissue has been liquefied and mixed with the tumescent fluid forming a homogenized tumescent fluid and adipose tissue solution. Presence of such low impedance variations means that a treatment end-point in the treated volume has been reached and the probe may be advanced to the next volume of adipose tissue to be treated. The method enables accurate determination of the treated tissue volume, the movement to the next volume to be treated, and variation of the size of the treated volume. (Treatment end-point determination may also be performed based on load cell readings, since liquefied adipose tissue has lower resistance to mechanical movement than non-liquefied adipose tissue.) The size of the treated or affected volume is varied by changing the distance between the electrodes 220 and 224 of the pair of electrodes. In course of the treatment particles of carbonized tissue may reside on the distal end of the probe 204. Charred blood and burned muscle particles may reside on the distal end of the probe 204 reducing the effectiveness of the treatment. In order to prevent these phenomena and in particular blood charring a cooling fluid is delivered by pump 144 (FIG. 1) to the distal end of the probe.

While the exemplary embodiment of the method of and apparatus for guiding a tissue treatment probe and determining its location in the tissue have been illustrated and described, it will be appreciated that various changes can be made therein without affecting the spirit and scope of the method. The scope of the method, therefore, is defined by reference to the following claims:

What is claimed is:

1. An apparatus for cosmetic and body shaping procedures, said apparatus comprising:
a probe having an elongated laser radiation conducting body the distal end of the body is operative to be inserted into a volume of tissue; said distal end is terminated by a at least one electrode, and wherein the proximal end of the probe is terminated by a connector operative to receive one of more types of RF voltages and laser radiation;
an external electrode configured to be operatively in permanent contact with skin surface and having an area substantially larger than the at least one electrode;
at least one source of RF voltage configured to provide at least one type of RF voltage between the at least one electrode and the external electrode and at least one source of laser radiation.

2. The apparatus according to claim 1, wherein the external electrode surface is substantially larger that the surface of the at least one electrode and wherein the external electrode is a flexible electrode or a grid of electrodes.

3. The apparatus according to claim 1 further comprising a mechanism for monitoring the impedance between the at least one electrode and the external electrode.

4. The apparatus according to claim 1 wherein the at least one electrode is a pair of electrodes and wherein a mechanism for monitoring the impedance also monitors the impedance between the electrodes of the pair of electrodes.

5. The apparatus according to claim 1, wherein the probe is operative to apply to the tissue at least one of the treatment energies of a group consisting of an RF voltage, an optical radiation, and a combination of both of them.

6. The apparatus according to claim 4 wherein the pair of electrodes terminating the probe are at least one of a group consisting of flat electrodes, cylindrical electrodes, semicircular electrodes, and concentric electrodes.

7. The apparatus according to claim 4 wherein the pair of electrodes located at the distant end of the probe are arranged to change the distance between them.

8. The apparatus according to claim 4, wherein the source of RF voltage is operative to apply high power RF voltage between the pair of electrodes located at the distal end of the probe.

9. The apparatus according to claim 4, further comprising a mechanism interpreting the changes in the impedance between the pair of electrodes to indicate the treatment process progress, the treatment end-point, and the temperature of the treated volume.

10. The apparatus according to claim 1, wherein further comprising a mechanism operative to indicate the type of tissue treated.

11. The apparatus according to claim 4 wherein the RF voltage supplied between the pair of electrodes located at the distal end of the probe and the external electrode has at least one different parameter, said parameter consisting at least of RF voltage amplitude or RF voltage frequency from the voltage supplied between the pair of electrodes located at the distal end of the probe.

12. The apparatus according to claim 1 further comprising a probe with a cooling fluid conducting channel configured to deliver the cooling fluid to the distal end of the probe.

13. The apparatus according to claim 1, wherein the source of RF voltage supplies the RF energy in a pulse mode or in a continuous mode and, wherein the source of laser radiation supplies the laser radiation in a pulse mode or in a continuous mode.

14. The apparatus according to claim 1 further comprising a load cell operative to measure forces acting on the probe.

15. An apparatus for cosmetic and body shaping procedures, said apparatus comprising:
a probe having an elongated laser radiation conducting body the distal end of the body is configured for insertion into a volume of tissue and terminated by a pair of electrodes and wherein the proximal end of the probe is terminated by a load cell and a connector operative to receive one of more types of RF voltages and laser radiation;
an external electrode operative to be in permanent contact with skin surface and having an area substantially larger than each of the pair of the electrodes;
at least one source of RF voltage configured to provide at least one type of RF voltage between the electrodes and at least one source of laser radiation.

16. The apparatus according to claim 15, wherein the external electrode surface is substantially larger that the surface of each of the pair of electrodes and wherein the external electrode is at least one of a group of electrodes consisting of a flexible electrode or a grid of electrodes.

17. The apparatus according to claim 15 further comprising a mechanism for monitoring the impedance between the at least one electrode of the pair of electrodes and the external electrode, and between the electrodes of the pair of electrodes.

18. The apparatus according to claim 15, wherein the probe is configured to apply to the tissue at least one of the treatment energies of a group consisting of an RF voltage, an optical radiation, and both of them.

19. The apparatus according to claim 15 wherein the pair of electrodes terminating the probe are at least one of a group consisting of flat electrodes, cylindrical electrodes, semicircular electrodes, and concentric electrodes.

20. The apparatus according to claim 15 wherein the pair of electrodes located at the distal end of the probe are arranged to change the distance between them.

21. The apparatus according to claim 15, wherein the probe is configured to apply between the pair of electrodes located at the distal end high power RF voltage.

22. The apparatus according to claim 15, further comprising a mechanism operative to interpret the changes in the impedance between the pair of electrodes to indicate the treatment process progress and the temperature of the treated volume.

23. The apparatus according to claim 15, wherein further comprising a mechanism configured to indicate the type of tissue treated.

24. The apparatus according to any one of claim 15 or 19 wherein the RF voltage supplied between the pair of electrodes located at the distal end of the probe and the external electrode has at least one different parameter consisting at least of RF voltage amplitude or RF voltage frequency from the voltage supplied between the pair of electrodes located on the distal end of the probe.

25. The apparatus according to claim 15 further comprising a probe with a cooling fluid conducting channel configured to deliver the cooling fluid to the distal end of the probe.

26. The apparatus according to claim 24, wherein the source of RF energy applies the RF energy in a pulse mode or in a continuous mode and, wherein the source of laser radiation applies the laser radiation in a pulse mode or in a continuous mode.

27. The apparatus according to claim 15, wherein the load cell is operative to measure forces acting on the probe.

28. A method for guiding a probe delivering tissue treatment energy in cosmetic and body shaping procedures, the method comprising:
providing a probe comprising at least a light guide terminated on a distal end of the probe and at least one electrode mounted on the distal end of the probe;
penetrating the distal end of the probe into a volume of adipose tissue to be treated and applying to it laser radiation, liquefying the adipose tissue, and forming a pass enabling easy probe into the tissue penetration; and
concurrently applying to the treated volume of the tissue a high power RF energy, liquefying a larger volume of the adipose tissue and coagulating blood vessels.

29. The method according to claim 28 further comprising:
providing at least one external electrode and applying it to a segment of skin surface such as to maintain a permanent contact between the electrode and the skin surface;
applying a low power RF signal between the at least one electrode and the external electrode and monitoring the impedance between the two electrodes.

30. The method according to any one of claims 28 and 29 wherein the variations in the impedance of the tissue located between the at least one electrode and the external electrode are used to guide the probe in the tissue volume.

31. The method according to claim 28 wherein a high impedance value indicates on probe location proximate to adipose tissue and a low impedance value indicates on probe location proximate to muscular tissue.

32. The method according to claim 28 wherein the variation from a high impedance value to a low impedance value indicates shift from adipose tissue to muscular tissue and the shift from low impedance value to high impedance value indicates shift from muscular tissue to adipose tissue.

33. The method according to claim 28, wherein the laser radiation is applied in a pulse mode or in a continuous mode.

34. The method according to claim 28, wherein the RF energy is applied in a pulse mode or in a continuous mode.

35. The method according to claim 28, wherein the laser radiation wavelength is selected to coagulate blood vessels in the treated tissue volume.

36. The method according to claim 28, wherein the at least one electrode is a pair of electrodes and wherein the pair of electrodes is maintained under equal RF potential.

37. The method according to claim 36 further comprising varying the volume of affected adipose tissue by changing the distance between the electrodes of the pair of electrodes.

38. The method according to claim 36, further comprising a mechanism operative to monitor the impedance between the pair of electrodes.

39. The method according to claim 36, further comprising introducing a tumescent fluid environment in the treated tissue volume and monitoring the impedance between the pair of electrodes.

40. The method according to claim 36, wherein sudden high magnitude variations of the impedance between the pair of electrodes located in the tumescent environment of the treated tissue volume indicate on presence of pockets of adipose tissue mixed with the tumescent fluid in the treated volume.

41. The method according to claim 36, wherein low magnitude variations of the impedance between the pair of electrodes located in the tumescent environment of the treated tissue volume indicate on the treatment end-point in the treated tissue volume.

42. The method according to claim 36, wherein the impedance between the pair of electrodes indicates the treated tissue volume temperature and the treated tissue aggregate state.

43. The method according to claim 28 further comprising preventing charring by cooling the distal end of the probe.

44. The method according to any one of claims 28 and 36 wherein the variations in the impedance measured between the at least one electrode and the external electrode are substantially lower for muscular tissue than for adipose tissue.

45. The method according to claim 28 further comprising providing a load cell and monitoring forces acting on the probe.

46. The method according to claim 45 wherein high forces acting on the probe indicate on probe location proximate to muscular tissue.

47. A method for determining a type of treated tissue in cosmetic and body shaping procedures, the method comprising:
providing a probe the distal end of which is terminated by at least one electrode;
introducing the probe into a volume of tissue to be treated and advancing it in said volume;
providing an external electrode having an area substantially larger than the at least one electrode and applying it to a segment of skin surface such as to maintain a permanent contact between the external electrode and the skin surface;
applying a low power RF signal between the at least one electrode and the external electrode and monitoring the impedance between at least one of the electrode and the external electrode; and
wherein the changes in the impedance of the tissue located between the at least one electrode and the external electrode indicate the type of the tissue treated.

48. The method according to claim 47 wherein high impedance measured between the at least one electrode and the external electrode indicates that the probe is applied to adipose tissue.

49. The method according to claim 47 wherein the at least one electrode is a pair of the electrodes maintained under an equal potential.

50. The method according to claim 49, wherein the impedance between the pair of the electrodes indicates the treatment process progress and the temperature of the treated volume, and wherein the temperature of the treated volume indicates the aggregate state of the treated volume of the tissue.

51. The method according to claim 49 further comprising injecting a tumescent fluid into the treated volume of the tissue and monitoring the impedance between the pair of electrodes and wherein magnitude of variations of the impedance between the pair of the electrodes indicates the treatment end-point.

52. The method according to any one of claims 49 and 50 further comprising comparing the impedance between the pair of electrodes and the external electrode and indicating the type of tissue treated.

53. The method according to claim 47, wherein the probe further comprises a light guide terminating at the distal end of the probe and wherein conducting laser energy through the light guide generates a pass to be easy penetrated by the distal end of the probe.

54. A method of safe tissue treatment by laser radiation, said method comprising:
providing a probe the distal end of which is terminated by a light guide and at least one electrode;
inserting the probe into a volume of tissue to be treated and applying to it laser radiation;
providing an external electrode and applying it to a segment of skin surface;
applying a low power RF signal between the at least one electrode located on the distal end of the probe and the external electrode and monitoring the impedance between the two electrodes; and
where in the changes in the impedance of the tissue located between the at least one electrode and the external electrode indicate the type of tissue treated by the laser radiation.

55. A method for guiding a probe delivering tissue treatment energy in a cosmetic and body shaping procedures, the method comprising:
providing a probe comprising at least a light guide terminated on a distal end of the probe and a pair of electrodes located at the distal end of the probe;
penetrating the distal end of the probe into a volume of adipose tissue to be treated and applying to it laser radiation, liquefying the adipose tissue, and forming a pass enabling easy probe into the tissue penetration;
concurrently applying to the treated volume of the tissue a high power RF energy, expanding the treated volume of the adipose tissue and liquefying the expanded volume of adipose tissue;
providing at least one external electrode and applying it to a segment of skin surface such as to maintain a permanent contact between the electrode and the skin surface;
applying a low power RF voltage between the at least one of the pair of electrodes and the external electrode and monitoring the impedance between the two electrodes; and
wherein the changes in the impedance of the tissue located between the at least one of the pair of electrodes and the external electrode guide the probe in the treated tissue volume.

56. A method of safe tissue treatment by laser radiation, said method comprising:
providing a probe the distal end of which is terminated by a light guide and at least one electrode;
inserting the probe into a volume of tissue to be treated and applying to it laser radiation;
providing an external electrode and applying it to a segment of skin surface;
applying a low power RF signal between the at least one electrode located on the distal end of the probe and the external electrode and monitoring the impedance between the two electrodes; and
wherein the changes in the impedance of the tissue located between the at least one electrode and the external electrode indicate the type of tissue treated by the laser radiation.

57. An apparatus for cosmetic and body shaping procedures, said apparatus comprising:
a probe having an elongated laser radiation conducting body the distal end of the body is operative to be inserted into a volume of tissue and a load cell operative to measure the tissue to mechanical movement resistance force; and
at least one source of laser radiation operatively configured to provide at least one type of laser radiation into the volume of tissue.

58. An apparatus for cosmetic and body shaping procedures, said apparatus comprising:
a probe having an elongated body the distal end of the body inserted in use into a volume of tissue and terminated by a at least one electrode, and wherein the proximal end of the probe is terminated by a load cell operative to measure mechanical forces acting on the probe;
an external electrode being in course of operation in permanent contact with skin surface and having an area substantially larger than the at least one electrode; and
at least one source of RF voltage configured to provide at least one type of RF voltage between the at least one electrode and the external electrode.

59. A method of safe tissue treatment by laser radiation and RF energy, said method comprising:
providing a probe the distal end of which is terminated by a light guide, a pair of electrodes, and the proximal end of the probe communicates with a load cell;
inserting the probe into a volume of tissue to be treated and applying to it laser radiation and RF energy;
moving the probe the volume of tissue and monitoring by the load cell forces acting on the probe; and
where in the changes in the forces acting on the probe indicate the type of tissue treated by the laser radiation and RF energy.

60. An apparatus for cosmetic and body shaping procedures, said apparatus comprising:
a probe having an elongated laser radiation conducting body the distal end of the body is operative to be inserted into a volume of tissue; said distal end is terminated by a pair of electrodes;
at least one source of laser radiation operative to provide at least one type of laser radiation into the volume of tissue; and
at least one source of RF voltage configured to provide at least one type of RF voltage between the at least one electrode and the external electrode and at least one source of laser radiation.

61. The apparatus according to claim 60 further comprising a probe a proximal end of which is terminated by a connector operative to receive one of more types of RF voltages and laser radiation.

62. The apparatus according to claim 61 wherein the proximal end of the probe includes a load cell.

63. A method of safe tissue treatment by laser radiation and RF energy, said method comprising:
providing a probe the distal end of which is terminated by a light guide and a pair of electrodes;
conducting through the probe laser radiation of a power sufficient to make an incision in the tissue and penetrating the probe into a volume of tissue to be treated, liquefying the adipose tissue, and forming a pass enabling easy probe into the tissue penetration; and
concurrently applying to the treated volume of the tissue a high power RF energy, liquefying a larger volume of the adipose tissue and coagulating blood vessels.

64. The method according to claim 63 further comprising moving the probe in the volume of tissue and monitoring by a load cell forces acting on the probe, and wherein the changes in the forces acting on the probe indicate the type of tissue treated by the laser radiation and RF energy.
